(19) United States
(12) Patent Application Publication
(10) Pub. No.: US 2007/0073103 A1
(43) Pub. Date: Mar. 29, 2007

(54) Diagnostic device for tubular organs

(76) Inventor: Naomichi Akizuki, Fukushima (JP)

Correspondence Address: LADAS & PARRY, 5670 WILSHIRE BOULEVARD, SUITE 2100, LOS ANGELES, CA, 90036-5679, US

(21) Appl. No.: 10/575,050

(22) PCT Filed: Oct. 6, 2003
(86) PCT No.: PCT/JP03/12767

§ 371 Date: Apr. 3, 2006

(51) Int. Cl. A61B 1/04

(52) U.S. Cl. 600/109

(57) ABSTRACT

In order to provide a diagnostic device for tubular organs that is capable of displaying or outputting an endoscopic image sequence of a inner wall of a tubular organ as continuous developed still images, the diagnostic device is provided with a developed still image generation means that is constituted by a pipe projection converting means used for forming a developed image of the inner wall of the tubular organ in the circumferential direction for each frame of the digital image data acquired in the diagnostic device and a mosaic processing means for cutting out strips of each frame of the developed images formed by the pipe projection converting means so that the strips are stitched to be converted to continuous developed still image data without stitched portions.

### BACKGROUND OF THE INVENTION

1. Field of the Invention

This invention relates to a diagnostic device for tubular organs (such as esophagus, stomach, small intestine, large intestine, rectum, ureter, urinary bladder, urethra, vagina, uteric tube, pancreatic duct, lacrimal gland, nasal cavity, auditory tube, lung, heart and kidney), and more particularly concerns the diagnostic device for tubular organs, which is capable of displaying or outputting an endoscopic image sequence of a pipe of a tubular organ as continuous developed still images (in which the pipe-shaped organ is cut open).

2. Description of the Related Art

In the medical field, upon diagnosing a tubular organ by using an endscope, images of the pipe of an affected part and the peripheral portion thereof are often picked up by a small-size CCD camera. The photographed images are displayed on a display as video images in one case, and in another case, one portion of the video frames is outputted and printed so as to be utilized as still images. In the former case, it is inconvenient to store the video images together with a carte and the like, and there is a disadvantage in that the positional relationship, for example, as to how far it is from the affected portion to the entrance of the organ, is not accurately confirmed. In the latter case, since continuous images are extracted as several partial photographs, it becomes difficult to confirm the entire image, and high technical knowledge and experiences are required so as to determine which portion of the entire organ a specific portion corresponds to. Moreover, since the number of photographs to be printed is limited, the vicinity of an affected portion tends to be photographed, with the result that the metastasis of cancer cells or the like might be ignored.

The common problem with the two cases is that since images are picked up while an endoscopic CCD camera is being transferred in the advancing direction (axis direction) of a tubular organ, the resulting images form doughnut-shaped images like images of a tunnel. Consequently, in order to obtain detailed images of the state of the pipe, it is necessary to pick up images with the CCD camera being rotated inside the pipe in the circumferential direction, and this method is not a practical method to be applied to the human from the viewpoint of pains of the patient.

Under these circumstances, it is very convenient if picked-up video images of an inner wall (tunnel-like images) of a tubular organ can be displayed or outputted and printed as continuous developed still images.

The present invention has been devised so as to solve the above-mentioned conventional problems, and its objective is to provide a diagnostic device for tubular organs, which is capable of displaying or outputting an endoscopic image sequence of an inner wall of a tubular organ as continuous developed still images (in which the pipe-shaped organ is cut open).

### SUMMARY OF THE INVENTION

The present invention provides a diagnostic device that generates continuous developed still images from video images of an inner wall of a tubular organ so that a diagnosis on a sickness is conducted based on the developed still images, and in order to achieve the objective, the diagnostic device includes: image-pickup means used for picking up video images of the inner wall of the tubular organ; digital image data acquiring means used for acquiring the picked-up video images as digital image data; developed still image generation means used for generating continuous developed still images without stitched portions from the acquired digital image data; and at least either one of display means for displaying the developed still images thus generated and output means for outputting printed images, and in this structure, the developed still image generation means includes a pipe projection converting means used for forming a developed image of the inner wall of the tubular organ in the circumferential direction for each frame of the acquired digital image data and a mosaic processing means for cutting out strips of each frame of the developed images formed by the pipe projection converting means so that the strips are stitched to be converted to continuous developed still image data without stitched portions.

Moreover, the above-mentioned objective of the present invention is achieved more effectively by preparing the image-pickup means as a cable-type endoscopic camera or a capsule-type radio small-size video camera.

### BRIEF DESCRIPTION OF THE DRAWINGS

FIG. 1 is a block diagram that shows the entire structure of a diagnostic device for tubular organs in accordance with the present invention.

FIG. 2 is a drawing that explains the concept of pipe projection.

FIG. 3 is a flow chart of processes used for forming developed still images, and FIG. 4 is a conceptual drawing thereof.

FIG. 5 is a drawing that explains the principle of a pipe projection conversion.

FIG. 6 is a drawing that explains forming processes of a developed image in which the pipe projection conversion is used.

FIG. 7 is one example of a computer flow chart used for forming a developed image by using the pipe projection convention.

### BEST MODE FOR CARRYING OUT THE INVENTION

Referring to Figures, the following description will discuss preferred embodiments of a diagnostic device for tubular organs in accordance with the present invention.

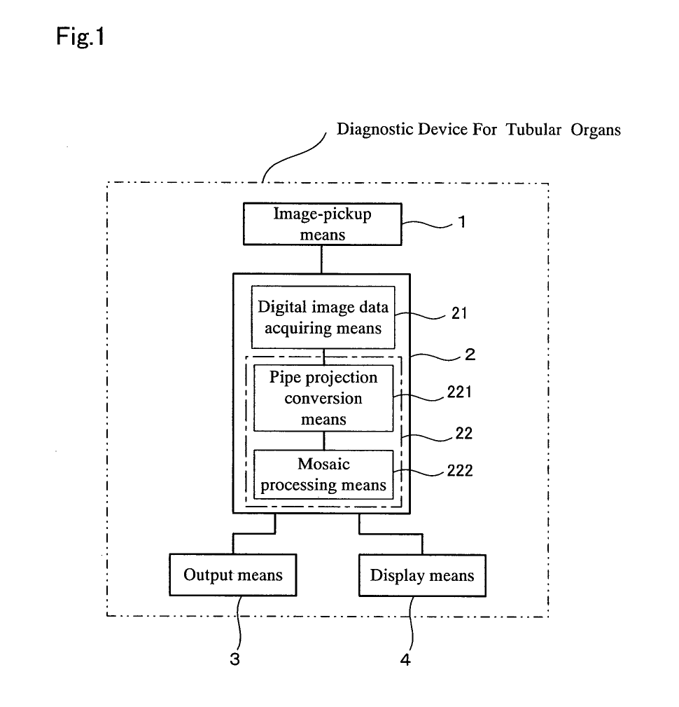

In FIG. 1, an image-pickup means 1 is used for shooting video images of the inner wall of a tubular organ, and prepared as an endscopic camera in which a small-size CCD is installed or as a capsule-type radio small-size video camera or the like. The former is operated by a doctor to pick up images, and the latter is swallowed by a patient so that images of the inner wall of a tubular organ are picked up through a remote-controlling operation, and the resulting data is transmitted to a diagnostic device main body 2, which will be described later, through radio. Here, the image data may be temporarily stored in a built-in memory of the capsule and taken out later.

With respect to the capsule-type radio small-size camera, those described in Japanese Patent Application Laid-Open No. 08-79589 may be utilized.

In FIG. 1, reference numeral 2 represents the main body of the diagnostic device which is provided with a digital image data acquiring means 21 and a developed still image generation means 22. The developed still image generation means 22 includes a pipe projection conversion means 221 and a mosaic processing means 222.

The digital image data acquiring means 21 is used for acquiring images picked up by the image pick-up means 1 into the diagnostic device main body 2 as digital image data, and in the case when the image pick-up means 1 is prepared as an endoscopic camera of a digital video camera type, for example, an IEEE1394 interface corresponds to this device, and the digital signals, as they are, can be taken in the device main body. Moreover, in the case when the image data picked up by the image pickup means 1 forms analog signals, for example, avideo capture board or the like is used for the digital image data acquiring means 21 so that the analog image data is converted to digital image data, and taken into the diagnostic device main body 2.

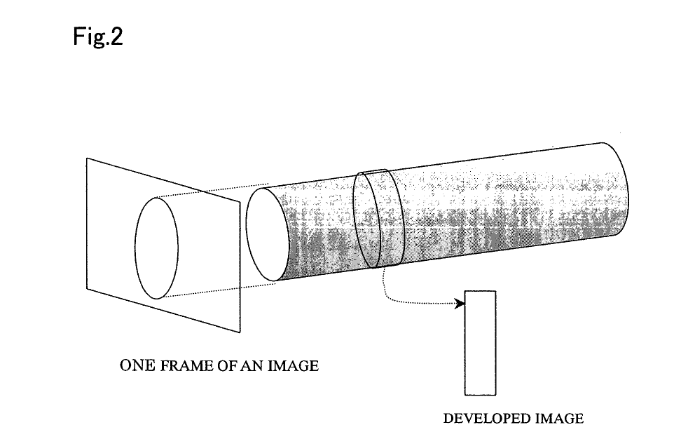

The digital image data, thus acquired, is pipe-projection converted for each frame of image data by the pipe production conversion means 221 so that developed images are obtained. Further, the mosaic processing means 222 cuts out strips (strip-like images) from the developed image of each of the above-mentioned frames, and stitches the strips so that continuous developed still images are obtained. Here, as shown in FIG. 2, the pipe projection refers to processes in which one frame of the image of the inner wall of a tubular organ is projected onto a three-dimensional cylinder (pipe) that intersects the face of the image so that one portion of the projected cylinder is developed into a rectangular image.

Here, a pipe projection conversion means and a mosaic processing means described in Japanese Patent Application Laid-Open No. 2003-32674 are optimally adopted as the pipe projection means 221 and the mosaic processing means 222 that constitute the developed still image generation means 22.

With respect to the developed still image generation means 22, the following description will discuss a step in which the pipe projection conversion is carried out on each frame of the digital image data to form developed images so that continuous developed still images are formed through the mosaic processing.

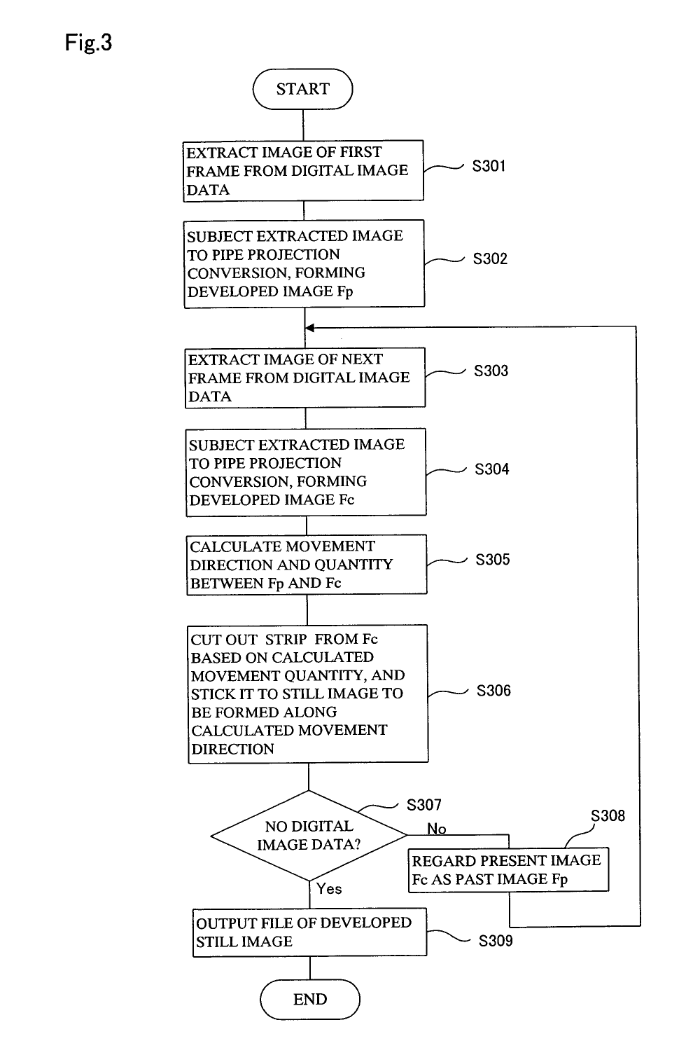

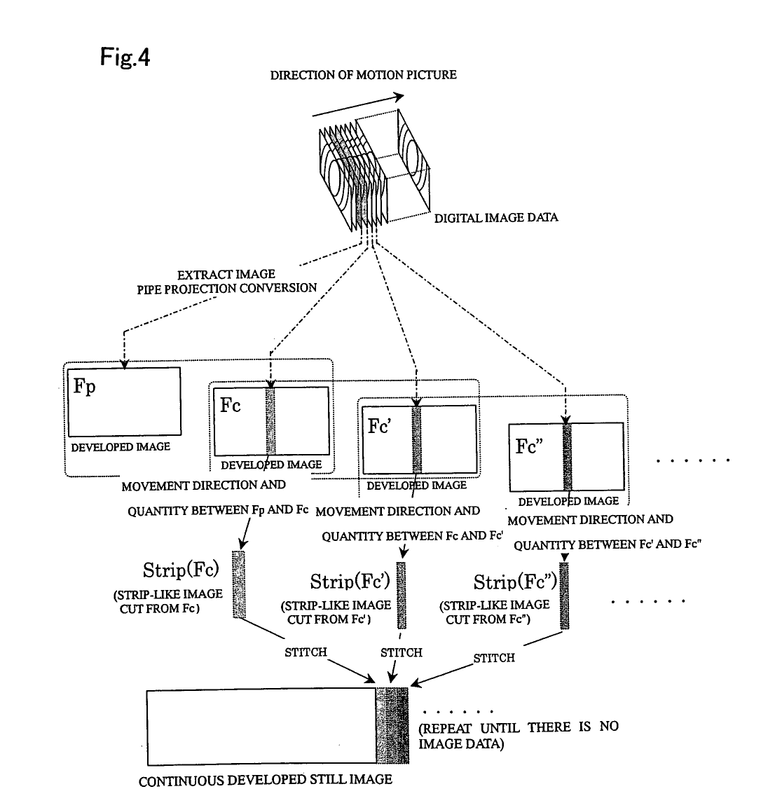

FIG. 3 is a flow chart of processes used for forming developed still images, and FIG. 4 is a conceptual drawing thereof.

First, at step S301, an image of a first frame is extracted from digital image data, and at step S302, the image thus extracted is subjected to a pipe projection conversion to form a developed image. This developed image is defined as Fp. The principle of the developed image formation is described below.

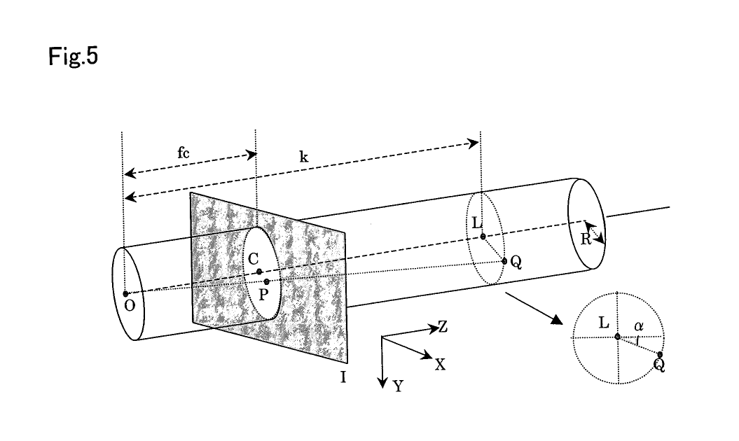

First of all, the projection of the image to the three-dimensional pipe is considered. The relationship between the image and the pipe is set as illustrated in FIG. 5. Reference character I designates the digital image obtained by photographing the inner wall of the pipe (tubular organ); and reference character R designates the radius of the pipe. The axis of the pipe is selected in such a manner as to pass through an optical center O and a focusing point C (cx, cy) of a camera (here, for the sake of simplification, it is construed that a Z axis in absolute coordinates is parallel to the axis of the pipe). Reference character fc designates a focal distance of the camera. Each image point P (x, y, fc) on the image I is projected on a corresponding point Q on the pipe. The point Q is aligned with the optical center O and the image point P. A point L is located as a projected point of the point Q on the axis of the pipe. Reference character k designates a distance between the point L and the optical center O. Moreover, α represents an angle formed between a line connecting the point L to the point Q and a line parallel to the X axis in the absolute coordinates and passing the point L. At this time, the point Q on the pipe can be expressed by the following equation (1):
Q(Qx, Qy, Qz)=(R cos α, R sin α, k) (1)

Additionally, the point P on the image screen with respect to the point Q can be expressed by the equation (2) below by using the focal distance fc and the distance k:
P(x, y, fc)=(fc/k*Qx, fc/k*Qy, fc) (2)

In unifying the above-described equations, the point P can be expressed by the equation (3) below with respect to fc, k, R and α.
P(x, y, fc)=(fc/k*R cos α, fc/k*R sin α, fc) (3)

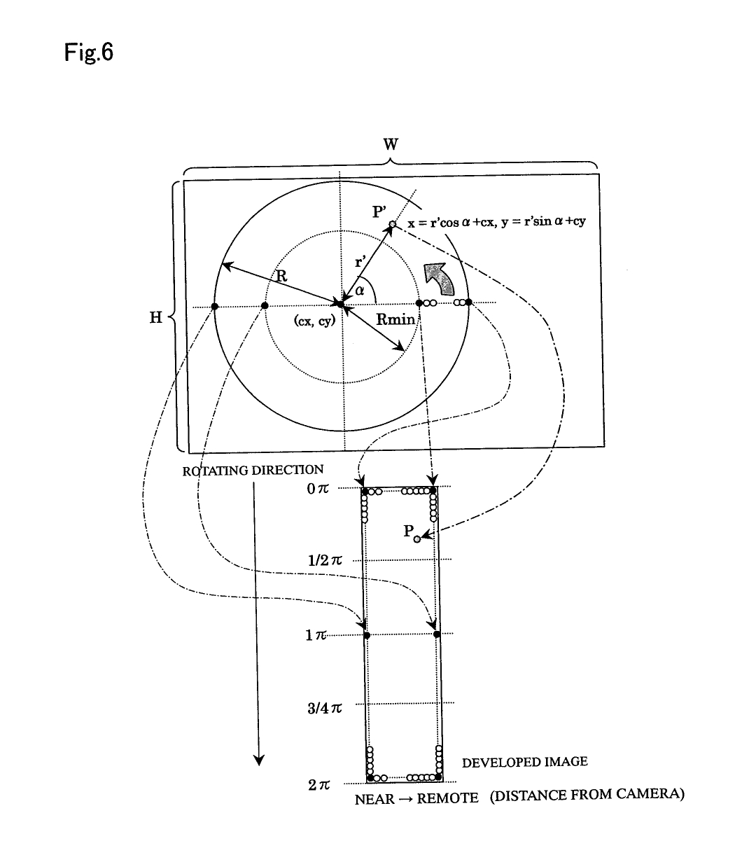

FIG. 6 illustrates an actually developed state. The width of the developed image is calculated in accordance with the following expression:
fc*(R−Rmin)/Rmin
by using the radius R and Rmin of the pipe reflected on the image I before the development and the focal distance fc (here, the radius Rmin refers to “the pipe minimum radius of an object to be developed”. In performing the pipe projection, the actual photographing distance becomes remote as the object to be developed is located nearer the center of the pipe, thereby reducing resolution at the time of the development. Consequently, it is necessary to develop the image by setting the radius Rmin to an appropriate value.). In addition, the height of the developed image is equal to the length of a circumference having the radius R of the pipe (i.e., 2πR). Here, if an arbitrary point on the developed image is represented by P(x, y), the pipe projection can be achieved by obtaining a point P′ on the image I corresponding to each of the points P, and copying pixel data on the point P′. A pixel at a left end of the developed image corresponds to a pixel on the circumference having the radius R of the image I; in contrast, a pixel at a right end of the developed image corresponds to a pixel on the circumference of the radius Rmin of the image I.

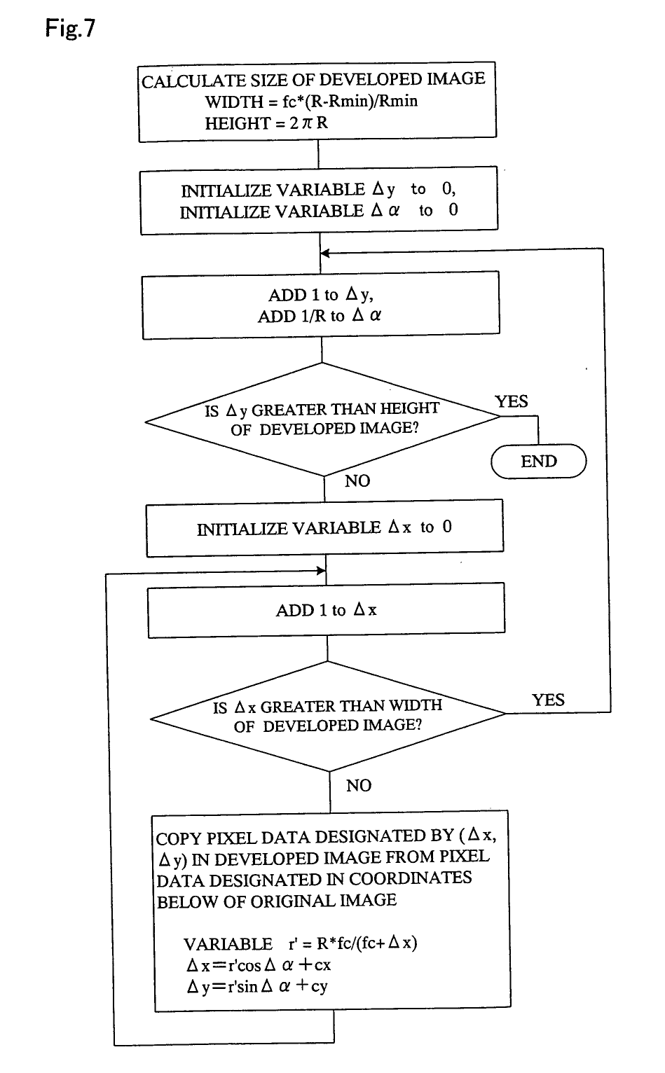

FIG. 7 is a flowchart illustrating one example of the pipe projection conversion processed by the use of the computer, although the explanation will be omitted below.

Subsequently, in step S303, an image of a second frame is extracted from the digital motion picture data. In step S304, the extracted image is subjected to the pipe projection conversion, thereby forming a developed image, which is designated by Fc.

Next, in step S305, the movement quantity and direction between the developed images Fp and Fc are calculated. Incidentally, although the movement quantity and direction between the developed images Fp and Fc are calculated by the use of algorithm in accordance with an “optical flow constraint equation” in the present preferred embodiment, the present invention is not restricted to such algorithm.

Subsequently, in step S306, a strip-like image (hereinafter referred to as a “strip”. Since the strip is cut from a portion of the image with least distortion, it is normally the center of the image.) is cut from the developed image Fc based on the calculated movement quantity, and is stuck to a still image to be formed along the calculated movement direction. This is referred to as the mosaic processing. The width of the strip to be cut becomes greater as the movement quantity is larger; in contrast, it becomes smaller as the movement quantity is smaller. The data in the movement direction is used for positioning when the preceding and following images are stuck to each other.

Next, the present developed image (i.e., the second image) Fc is referred to as the developed image Fp (step S308). The control routine returns to step S303, in which an image of a third frame is extracted. In step S304, the extracted image is subjected to the pipe projection conversion, thereby forming a developed image, which is designated by Fc.

Subsequently, in step S305, in the same manner, the movement quantity and direction between the developed images Fp and Fc are calculated.

Next, in step S306, a strip is cut from the developed image Fc based on the calculated movement quantity, and then, is stuck to the immediately preceding strip along the calculated movement direction.

Hereinafter, the control routine is repeated until there is no image data.

Here, the diagnostic device main body 2 may be formed by exclusively-used hardware, or may be constituted by installing an exclusively-used pipe projection conversion program and mosaic processing program in a general-use personal computer.

The continuous developed still images of the inner wall of the tubular organ, generated by the developed still image generation means 22, are printed and outputted from the output means 3, or displayed as images on a display means 4. With respect to the embodiment of the output means 3, a printer may be used, and those of a type capable of printing images on continuous roll paper are optimally used, or those of cut-paper printing type may also be utilized. With respect to the embodiment of the display means 4, a color display or the like of a personal computer is optimally utilized.

### INDUSTRIAL APPLICABILITY

As described above, in accordance with the diagnostic device for tubular organs of the present invention, the affected portion can be viewed not as stitched partial photographs of the pipe of a tubular organ, but as a sheet of continuous developed images; therefore, the positional relationship of the affected portion and the entire tubular organ is easily confirmed so that an explanation to the patient can be easily made. Moreover, since portions other than the affected portion, such as metastasis of cancer cells, can also be sufficiently observed, oversights can be reduced, thereby making it possible to easily detect sickness earlier.

What is claimed is:

1. A diagnostic device for tubular organs, which generates continuous developed still images from video images of a inner wall of the tubular organ so that a diagnosis on a sickness is conducted based on the developed still images, comprising:
image-pickup means used for picking up video images of the inner wall of the tubular organ;
digital image data acquiring means used for acquiring the picked-up video images as digital image data;
developed still image generation means used for generating continuous developed still images without stitched portions from the acquired digital image data; and
at least either one of display means for displaying the developed still images thus generated and output means for outputting printed images,
wherein: the developed still image generation means comprises a pipe projection converting means used for forming a developed image of the inner wall of the tubular organ in the circumferential direction for each frame of the acquired digital image data; and a mosaic processing means for cutting out strips of each frame of the developed images formed by the pipe projection converting means so that the strips are stitched to be converted to continuous developed still image data without stitched portions.

2. The diagnostic device for tubular organs according to claim 1, wherein the image-pickup means is prepared as a cable-type endoscopic camera.

3. The diagnostic device for tubular organs according to claim 1, wherein the image-pickup means is prepared as a capsule-type radio small-size video camera.
